(19) United States
(12) Patent Application Publication
(10) Pub. No.: US 2004/0077027 A1
(43) Pub. Date: Apr. 22, 2004

(54) Assays and kits for detecting and monitoring heart disease

(76) Inventor: Leong Ng, Leicester (GB)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/618,567

(22) Filed: Jul. 11, 2003

(30) Foreign Application Priority Data
Jul. 11, 2002 (GB) 0216191.7
Jul. 16, 2002 (GB) 0216500.9
Jul. 17, 2002 (GB) 0216505.8

(51) Int. Cl. G01N 33/567

(52) U.S. Cl. 435/7.21

(57) ABSTRACT

The present invention provides a method for detecting an increased risk of heart failure in a subject by detecting an increased level of urotensin II in a bodily fluid sample from a subject

### BACKGROUND OF THE INVENTION

Heart failure (left ventricular systolic dysfunction) is a leading cause of mortality and morbidity. Heart failure affects millions of people worldwide and is the leading cause of death in the United States. Its pathophysiology involves activation of many neurohormonal systems, including the catecholamine, renin-angiotensin, endothelin, atrial and brain natriuretic peptide systems. Some of these systems are activated in an adaptive fashion, others are maladaptive.

Recent work has revealed the existence of a novel cardiovascular peptide called urotensin II (UTN) with homology to the hormone of teleosts (Ames et al., Nature 1999; 16: 282-286). This cyclic undecapeptide is the ligand for an orphan G-protein receptor (GPR14) and both peptide and receptors are distributed within the myocardium, endothelium, vascular myocytes and nervous system (Ames et al., Nature 1999; 16: 282-286). Although a potent vasoconstrictor for certain vascular beds in rats and monkeys (Ames et al., Nature 1999; 16: 282-286; Douglas et al J Cardiovasc Pharmacol 2000; 36: S163-6), it may be a vasodilator in the mesenteric resistance vessels (Bottrill et al. Br J Pharm 2000; 130: 1865-1870). There are important species differences in the reactivity of different vessels to UTN. For example, although monkey vessels are potently vasoconstricted by UTN, human pulmonary vasculature and mesenteric resistance vessels are vasodilated with high potency by UTN (Stirrat et al. Am J Physiol 2001; 280: H925-928) and human subcutaneous resistance vessels show no reactivity to UTN (Hillier et al. Circulation 2001; 103: 1378-1381). Chronic stimulation of vascular smooth muscle by UTN leads to hypertrophic responses (Watanabe et al. J Hypertens 2001; 19: 2191-2196). Direct effects on the myocardium were suggested in monkeys with myocardial depression (Ames et al., Nature 1999; 16: 282-286) probably resulting from coronary vasoconstriction, although in human heart muscle, a positive inotropic effect was demonstrated (Russell et al. Br J Pharm 2001; 132: 5-9). In addition, hypertrophic effects including increased collagen deposition have been described, suggesting a possible role for UTN in ventricular remodeling of heart failure (Zou et al. FEBS Lett 2001; 508: 57-60; Tzanidis et al. Eur Heart J 2000: 21: 72). UTN led to increased expression of the atrial and brain natriuretic peptides in cardiomyocytes (Zou et al. FEBS Lett 2001; 508: 57-60), a change expected in heart failure with reversion to a more primitive phenotype. Recent work by Douglas et al (Douglas et al. Lancet 2002; 359: 1990-1997) examined the expression of UTN and its receptor, GPR14 (or UT receptor) in myocardium of patients at various stages of congestive heart failure (CHF). UTN was found to be strongly expressed in cardiomyocytes, vascular smooth muscle cells, endothelium and inflammatory cells within the myocardium of patients with CHF. With regard to a putative role for urotensin in heart failure, the initial positive inotropic and vasodilator effect in humans may be compensatory, before remodeling and increased fibrosis leads to an increasingly maladaptive response.

Current diagnostic procedures for heart failure generally assess the extent of cardiac tissue damage after clinical signs have appeared. Current methods of identifying and confirming heart failure require more time than is often available in emergency situations where rapid evaluation is critical for effective patient treatment and survival. In an emergency medical facility, electrocardiography (ECG) monitoring of suspected patients is the most rapid diagnostic method for detecting heart failure (Mair et al., 1995).

Electrocardiography and currently available diagnostic blood tests are generally not effective for early detection of heart failure that precedes the damage associated with heart attacks because the tests detect infarction-associated tissue damage. They are not effective in early detection of heart disease. Currently, the only diagnostic for chronic underlying coronary artery disease is ECG monitoring during exercise stress (e.g., treadmill exercise). ECG is generally used to confirm the clinical symptoms of angina (chest pain). Such stress testing is usually given after the patient has experienced symptoms and sought treatment (e.g., at an emergency medical facility). Although stress testing is sometimes used to screen asymptomatic patients, testing is costly, time-consuming and generally not amenable to routine screening of large numbers of patients. Furthermore, exercise stress test evaluations result in about 15% false negatives.

Diagnostics tests have been developed that use cardiac proteins to determine whether or not the source of the patient's chest pain is cardiac and if so, whether the patient has suffered a myocardial infarct or is suffering from unstable angina (see, e.g., U.S. Pat. Nos. 5,290,678, 5,604,105, 5,710,008). Other diagnostic tests use non-polypeptidic cardiac markers for the early detection of heart disease (see U.S. Pat. No 6,534,322).

Accordingly, there remains a need for a better non-invasive, more sensitive, and highly reliable point-of-care ‘bedside test’ for the early detection of heart failure, particularly for people at risk for heart disease.

### SUMMARY OF THE INVENTION

The present invention is based on the finding that the levels of urotensin II in bodily fluids are raised in patients at increased risk for heart failure. The present invention as disclosed herein provides patients with a sensitive and reliable assay and kit to detect an increased risk of heart failure.

In a first aspect, the present invention provides methods for determining an increased risk of heart failure by detecting an increased level of urotensin II in a sample of bodily fluid.

In a preferred embodiment, the level of at least one further marker indicative of heart failure, such as N-terminal pro-Brain Natiuretic Peptide (NT-proBNP) or Brain Natiuretic Peptide (BNP) is measured.

The level of urotensin II alone or in combination with another marker is preferably determined by use of an immunoassay, and the bodily fluid is preferably plasma or interstitial fluid.

In a second aspect, the present invention provides kits for detecting the relative amount of urotensin II in a sample of bodily fluid incorporating the method of the first aspect.

Other features and advantages will be appreciated based on the following Detailed Description and claims.

### BRIEF DESCRIPTION OF THE DRAWINGS

FIG. 1 contains Table 1 which shows patient characteristics (Medians [ranges] are reported and P values were computed using the Kruskal-Wallis or Mann Whitney tests (comparing normal and heart failure patients).

FIG. 2 are graphs showing plasma NT-pro Brain Natriuretic Peptide (BNP) and plasma Urotensin respectively according to New York Heart Association class;

FIG. 3 are a receiver operating curves (ROC) for NT pro Brain Natriuretic Peptide and Urotensin respectively in the diagnosis of heart failure; and

FIG. 4 is a ROC curve prognostic index for combination of N-terminal pro Brain Natriuretic Peptide and Urotensin in the diagnosis of heart failure.

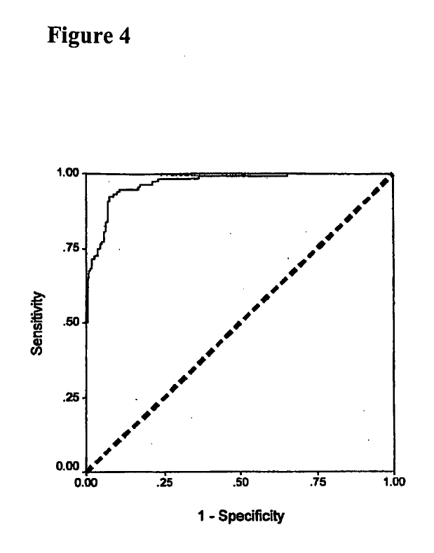

### DETAILED DESCRIPTION OF THE INVENTION

1. General

The invention provides assays and kits for detecting an increased risk of heart failure in a subject by detecting the level of urotensin II alone or in combination with another marker in a bodily fluid sample whereby an elevated level of urotensin II relative to the normal level is indicative of an increased risk of heart failure.

2. Definitions

For convenience, before further description of the present invention, certain terms employed in the specification, examples, and appended claims are collected here.

The singular forms “a”, “an”, and “the” include plural references unless the context clearly dictates otherwise.

“ANP” refers to atrial natriuretic peptide, the first described peptide in a family of hormones which regulate body fluid homeostasis (see. Brenner et al., Physiol. Rev.1990; 70: 665).

The term “antibody” as used herein refers to binding molecules including immunoglobulin molecules and immunologically active portions of immunoglobulin molecules, i.e., molecules that contain an antigen binding site that specifically bind an antigen. The immunoglobulin molecules useful in the invention can be of any class (e.g., IgG, IgE, IgM, IgD and IgA) or subclass of immunoglobulin molecule. Antibodies includes, but are not limited to, polyclonal, monoclonal, bispecific, humanised and chimeric antibodies, single chain antibodies, Fab fragments and F(ab′) fragments, fragments produced by a Fab expression library, anti-idiotypic (anti-Id) antibodies, and epitope-binding fragments of any of the above.

The term “bodily fluid” includes all fluids obtained from a mammalian body, including, for example, blood, plasma, urine, lymph, gastric juices, bile, serum, saliva, sweat, and spinal and brain fluids. Furthermore, the bodily fluids may be either processed (e.g., serum) or unprocessed.

“CNP” refers to C-type natriuretic peptide. (Stingo et al., Am. J. Physiol., 1992; 263,:H1318).

“Comprise” and “comprising” are used in the inclusive, open sense, meaning that additional elements may be included.

The term “heart failure” as used herein refers to the inability of the heart to keep up with the demands on it and, specifically, failure of the heart to pump blood with normal efficiency. Heart failure may result from coronary artery disease leading to heart attacks and heart muscle weakness, primary heart muscle weakness from viral infections or toxins such as prolonged alcohol exposure, heart valve disease causing heart muscle weakness due to too much leaking of blood or heart muscle stiffness from a blocked valve, and hypertension (high blood pressure). Rarer causes include hyperthyroidism (high thyroid hormone), vitamin deficiency, and excess amphetamine (“speed”) use. Other causes of heart failure may include ischaemic cardiomyopathy, dilated cardiomyopathy, hypertensive cardiomyopathy, valvular disease.

As used herein, an “immunoassay” is an assay that utilizes an antibody to specifically bind to a marker.

The term “marker level” as used herein refers to the amount of marker in a sample of bodily fluid or a mammalian subject and refers to units of concentration, mass, moles, volume, concentration or other measure indicating the amount of marker present in the sample.

As used herein, the term “natriuretic peptide” includes a native ANP, BNP, or CNP, portions of, variants of, or chimeras thereof.

“NT-proBNP” or “BNP” refers to cardiac derived peptide hormone that circulates in the blood and exerts potent cardiovascular and renal actions. Mature hBNP consists of a 32 amino acid peptide containing a 17 amino acid ring structure formed by two disulfide bonds.

The term “NYHA classification” refers to the New York Heart Association (NYHA) classification. This is a four-stage classification where:

Class 1. Patients exhibit symptoms only at exertion levels

Class 2. Patients exhibit symptoms with ordinary exertion.

Class 3. Patients exhibit symptoms with minimal exertion.

Class 4 Patients exhibit symptoms at rest.

A “subject” refers to a human or a non-human animal.

“Urotensin” or “UTN” as used herein refers to urotensin II (For example, polypeptide sequences of urotensin may be obtained from GenBank Accession Number NM_{—}021995, NM_{—}006786, O95399, and CAB63148). The term urotensin II also includes portions of, variants of, or allelic variants thereof.

3. Methods of Diagnosing Heart Failure

In the present invention, the measured level of urotensin (and, where measured, other marker(s) indicative of heart disease) is compared with a normal level. The normal level may be the level of urotensin (or a second marker) typically found in the bodily fluid of a subject which is indicative of the absence of heart failure. These normal levels may be determined from population studies of subjects free from heart failure. In one embodiment, the normal level may be determined when the particular subject is stabilised or is not suffering from heart failure or is suffering from less severe heart failure. This allows the relative changes of the marker(s) in the subject to be determined.

The levels of urotensin may be measured from a bodily fluid sample, such as blood, urine, lymph, and saliva, although any other body fluid, such as serum, gastric juices, and bile may be used. Methods of obtaining a bodily fluid sample from a subject are known to those skilled in the art.

In the present invention, the measured level of urotensin (and, where measured, other marker(s) indicative of heart disease) is compared with a normal level. The normal level may be the level of urotensin (or further marker) typically found in the bodily fluid which is indicative of the absence of heart failure. These normal levels may be determined from population studies of subjects free from heart failure. In one embodiment, the normal values of the levels of urotensin typically found in a sample of bodily fluid which is indicative of the absence of heart failure may range from 3-10 fmol/ml. Where measured, the normal values of NT-proBNP that is indicative of the absence of heart failure may range from 10-50 fmol/ml. These subjects may be matched for age and/or gender. Levels of urotensin that are indicative of an increased risk of heart failure may range from 10-15 fmol/ml, 15-20 fmol/ml, 20-25 fmol/ml, 25-30 fmol/ml or more. Levels of NT-proBNP that are indicative of an increased risk of heart failure may range from 300-600 fmol/ml, 600-1200 fmol/ml, 1200-1800 fmol/ml, 1800-2400 fmol/ml, 2400-3000 fmol/ml, 3000-3600 fmol/ml or more.

The immunoassay may comprise of an antibody or portion thereof sufficient for binding specifically to urotensin. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions. Antibodies can be naturally-occurring antibodies, e.g., monoclonal antibodies obtained by the method of Koehler and Milstein and polyclonal antibodies obtained, e.g., by injection of an antigen into an animal. Antibodies can also be partially or fully humanized antibodies, single chain antibodies or other variants of antibodies.

Antibodies binding to urotensin can be obtained commercially. Examples of commercially available antibodies binding to urotensin include anti-urotensin (Phoenix Pharmaceuticals), rabbit anti-urotensin (Biodesign International), rabbit anti-human urotensin (Immundiagnostik).

As mentioned, other markers that can be used in detecting an increased risk of heart failure may include N-terminal proBNP. Alternatively or additionally, the level of BNP may be measured. The release of stored proBNP (the intact precursor to the two circulating forms, BNP (the active peptide) and N-terminal BNP (NTproBNP the inactive peptide) from cardiac myocytes in the left ventricle and increased production of BNP is triggered by myocardial stretch, myocardial tension, and myocardial injury. In further embodiments, the second marker may be another natiuretic peptide, such as atrial natiuretic peptide (ANP) and/or its inactive form, N-terminal proANP (NTproANP) (Hall, Eur J Heart Fail, 2001, 3:395-397). In other embodiments, the second marker may be CNP which functions as a vasodilating and growth-inhibiting peptide (Suga et al., J Clin, Invest., 1992, 90:1145; Stingo et al., Am. J. Physiol., 1992, 262:H308; Stingo et al., Am. J. Physiol., 1992, 263:H1318; Koller et al., Science, 1991, 252:120). Other secondary markers that could be used to diagnose heart failure may include non-polypeptidic cardiac markers such as sphingolipid, sphingosine, sphingosine-1-phosphate, dihydrosphingosine and sphingosylphosphorylcholine (see U.S. Pat. No. 6,534,322). When measuring the levels of the above natiuretic peptides, non-natiuretic peptides, or non-poplypeptidic cardiac markers, corrections for age and gender may be necessary in order to improve the accuracy of diagnosis.

Antibodies binding to BNP and ANP can be obtained commercially. Examples of commercially available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological). Examples of commercially available antibodies binding to CNP include rabbit anti-C-Type Natriuretic Peptide-22 (Phoenix Pharmaceuticals).

Depending on the assays used to diagnose heart failure (see below), the antibodies specific to the markers of heart failure may further comprise a label, e.g., a fluorescent or magnetic label. In such embodiments, the antibody is said to be “directly labelled.” An antibody can also be “indirectly labelled,” i.e., the label is attached to the antibody through one or more other molecules, e.g., biotin-streptavidin. Alternatively, the antibody is not labelled, but is later contacted with a binding agent after the antibody is bound to a specific marker of heart failure. For example, there may be a “primary antibody” and a second antibody or “secondary antibody” that binds to the Fc portion of the first antibody. Labels may be linked, preferably covalently, to antibodies according to methods known in the art.

Further depending on the assays used to diagnose heart failure, antibodies may be linked to a solid surface. The solid surface can be selected from a variety of those known in the art including plastic tubes, beads, microtiter plates, latex particles, magnetic particles, cellulose beads, agarose beads, paper, dipsticks, and the like. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the antibodies with dithiothreitol followed by the addition of biotin maleimide.

Antibodies are preferably contacted with the sample of bodily fluid obtained from a mammalian subject at least for a time sufficient for the antibody to bind to a marker used to diagnose heart failure. For example, an antibody may be contacted with the sample of bodily fluid for at least about 10 minutes, 30 minutes, 1 hour, 3 hours, 5 hours, 7 hours, 10 hours, 15 hours, or 1 day.

The markers measured in the present invention may be detected using an immunoassay. In one embodiment, an immunoassay is performed by contacting a sample from a subject to be tested with an appropriate antibody under conditions such that immunospecific binding can occur if the marker is present, and detecting or measuring the amount of any immunospecific binding by the antibody. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a marker can be detected in a fluid sample by means of a two-step sandwich assay. In the first step, a capture reagent (e.g., an anti-marker antibody) is used to capture the marker. The capture reagent can optionally be immobilised on a solid phase. In the second step, a directly or indirectly labelled detection reagent is used to detect the captured marker. In one embodiment, the detection reagent is an antibody. In another embodiment, the detection reagent is a lectin. Any lectin can be used for this purpose that preferentially binds to the marker rather than to other proteins that share the antigenic determinant recognised by the antibody. In a preferred embodiment, the chosen lectin binds to the marker with at least 2-fold, 5-fold or 10-fold greater affinity than to other proteins that share the antigenic determinant recognised by the antibody. A lectin that is suitable for detecting a given marker can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174.

In one embodiment, a lateral flow immunoassay device may be used in the ‘sandwich’ format wherein the presence of sufficient marker in a bodily fluid sample will cause the formation of a ‘sandwich’ interaction at the capture zone in the lateral flow assay. The capture zone as used herein may contain capture reagents such as antibody molecules, antigens, nucleic acids, lectins, and enzymes suitable for capturing urotensin and other markers described herein. The device may also incorporate one or more luminescent labels suitable for capture in the capture zone, the extent of capture being determined by the presence of analyte. Suitable labels include fluorescent labels immobilised in polysterene microspheres. Microspheres may be coated with immunoglobulins to allow capture in the capture zone.

Other assays that may be used in the methods of the invention include, but are not limited to, flow-through devices.

In a flow-through assay, one reagent (usually an antibody) is immobilized to a defined area on a membrane surface. This membrane is then overlaid on an absorbent layer that acts as a reservoir to pump sample volume through the device. Following immobilization, the remainder of the protein-binding sites on the membrane are blocked to minimize nonspecific interactions. When the assay is used, a bodily fluid sample containing a marker specific to the antibody is added to the membrane and filters through the matrix, allowing the marker to bind to the immobilized antibody. In an optional second step (in embodiments wherein the first reactant is an antibody), a tagged secondary antibody (an enzyme conjugate, an antibody coupled to a colored latex particle, or an antibody incorporated into a colored colloid) may be added or released that reacts with captured marker to complete the sandwich. Alternatively, the secondary antibody can be mixed with the sample and added in a single step. If a marker is present, a colored spot develops on the surface of the membrane.

4. Kits

The invention also provides a kit for the detection of urotensin and other markers useful in detecting an increased risk of heart failure. Such a kit may comprise an antibody which binds specifically to urotensin and optionally another antibody which binds a second marker of heart failure. In addition, such a kit may optionally comprise one or more of the following: (1) instructions for using the kit for diagnosis of heart failure; (2) a labelled antibody or optionally, a labelled binding partner to the antibody; (3) a solid phase (such as a reagent strip) upon which each antibody is immobilised; and (4) a label or insert indicating regulatory approval for diagnostic, prognostic or therapeutic use or any combination thereof. If no labelled binding partner to each antibody is provided, each antibody itself can be labelled with a detectable marker, e.g., a chemiluminescent, enzymatic, fluorescent, or radioactive moiety. Additional antibodies to other markers of heart failure may be included in the kit.

### EXEMPLIFICATIONS

The invention, having been generally described, may be more readily understood by reference to the following examples, which are included merely for purposes of illustration of certain aspects and embodiments of the present invention, and are not intended to limit the invention in any way.

### Example 1

### Plasma Samples From Normal Controls and Patients with Heart Failure

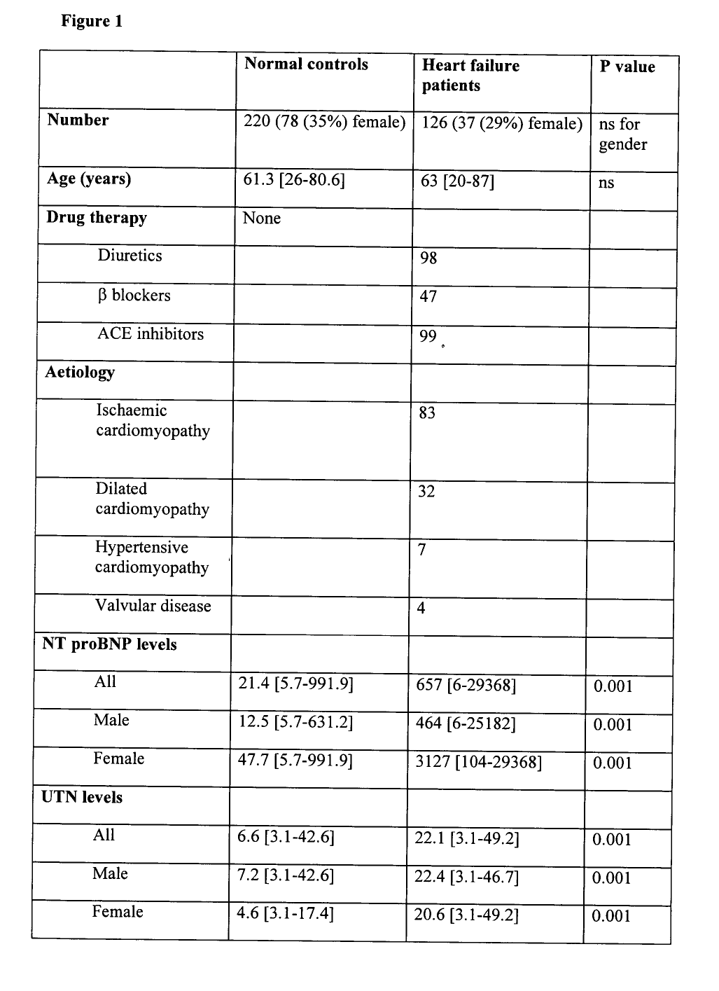

Patients with heart failure were recruited from the Leicester Royal Infirmary clinics and wards. All had a clinical diagnosis of heart failure and echocardiographically confirmed ejection fractions below 45%. Normal controls were age and gender matched with these patients, were on no medication and had echocardiographically confirmed ejection fractions greater than 55%. Patient characteristics are reported in FIG. 1.

10 mls of blood was obtained by venepuncture after 15 min bed rest and mixed in ice-cold tubes containing EDTA and aprotinin. Plasma recovered following centrifugation was stored at −70° C. until assayed.

### Example 2

### Assay of N-Terminal proBNP

The assay for N-terminal proBNP was based on the non-competitive N-terminal proBNP assay described by Karl (Karl et al. Scand J Clin Lab Invest Suppl 1999;230:177-181). Rabbit polyclonal antibodies were raised to the N-terminal (amino acids 1-12) and C-terminal (amino acids 65-76) of the human N-terminal proBNP. IgG from the sera was purified on protein A sepharose columns. The C-terminal directed antibody (0.5 μg in 100 μL for each ELISA plate well) served as the capture antibody. The N-terminal antibody was affinity purified and biotinylated. Aliquots (20 μL) of samples or N-BNP standards were incubated in the C-terminal antibody coated wells with the biotinylated antibody for 24 hours at 4° C. Following washes, streptavidin labelled with methyl-acridinium ester (streptavidin-MAE, 5×10⁶ relative light units/ml) (Hart & Taaffe, J Immunol Methods 1987;101: 91-96) was added to each well. Plates were read on a Dynatech MLX Luminometer as previously described (Hughes et al. Clin Sci 1999; 96: 373-380). The lower limit of detection was 5.7 fmol/ml of unextracted plasma. Within and between assay coefficients of variation were acceptable at 2.3% and 4.8% respectively. There was no cross-reactivity with ANP, BNP or CNP.

### Example 3

### Assay of Urotensin II

Antibody specific for the cyclic form of UTN was obtained from Phoenix Pharmaceuticals Inc., Belmont, Calif. Biotinylated UTN purified on reverse phase HPLC served as the tracer. A competitive assay using C₁₈ extracts of plasma was utilized, incubating 50 ng of the antibody with extracts or standards (ranging from 1 to 2000 fmol per well) in 100 μl of assay buffer (as described in 12). After 24 h of incubation at 4° C., the biotinylated UTN tracer was added (250 fmols per well). Immunoprecipitates were recovered in ELISA plates coated with anti-rabbit IgG (100 ng/well). Following washes and incubation with streptavidin-MAE, chemiluminescence was elicited as described above. Intra- and interassay coefficients of variation were 2.3 and 8.1% respectively, with no reactivity for BNP or N-terminal proBNP. The lower limit of detection was 3.1 fmol/ml.

Statistical analyses were performed on SPSS Version 11. Data are presented as medians [ranges]. Comparisons were by Kruskal-Wallis analysis of variance and receiver operating characteristic (ROC) curves were plotted. Correlation analysis employed Spearman's rho (rₛ). A value of P<0.05 was considered statistically significant.

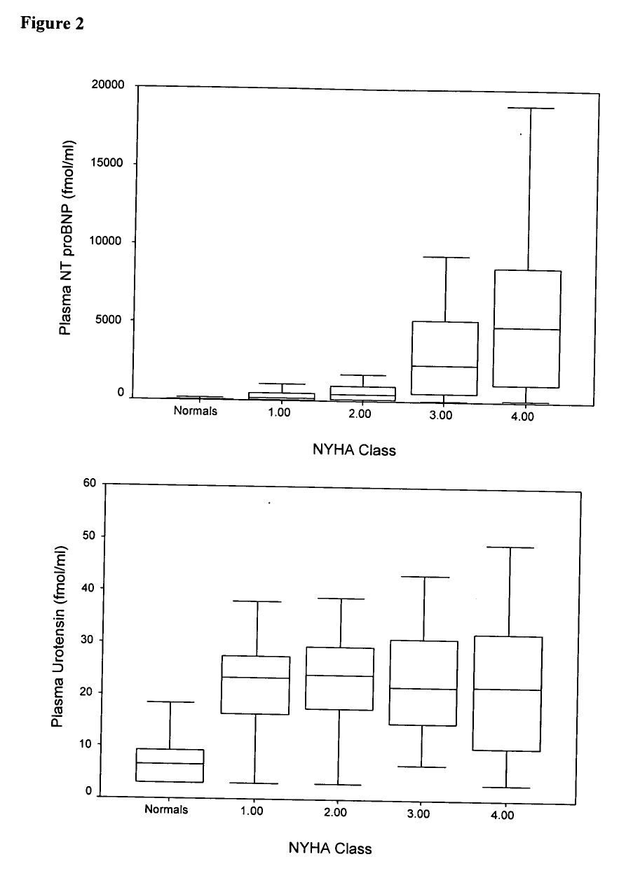

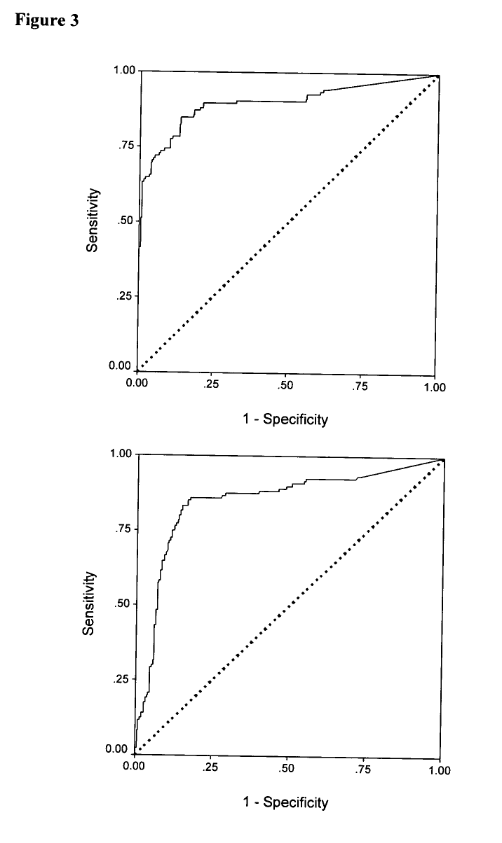

The results from this experiment are shown in FIG. 1 which illustrates the characteristics of the normal and heart failure patients, who were well matched for age and gender. As expected, N-terminal proBNP was significantly elevated in heart failure patients. In the normal population, there was a positive correlation of N-terminal proBNP with increasing age (rₛ=0.41, P<0.001) and females had higher levels than males (P<0.001, Table 1). N-terminal proBNP increased with increasing NYHA class (FIG. 1a, P<0.001 by Kruskal Wallis test). Plasma UTN was also elevated in heart failure patients (FIG. 1), but there was no correlation with age. In contrast to N-terminal proBNP, levels were lower in females compared to males (P<0.001, FIG. 1). Plasma UTN was not affected by increasing NYHA class (FIG. 2). Both N-terminal proBNP and UTN were elevated in heart failure patients irrespective of gender (P<0.001 for all comparisons). N-terminal proBNP and UTN were also modestly correlated (rₛ=0.35, P<0.001). In the heart failure patients, plasma UTN levels were not dependent on use of diuretics, beta blockers or ACE inhibitors. As shown in FIG. 3, ROC curves for the detection of heart failure for both peptides revealed areas of 0.90 and 0.86 for N-terminal proBNP and UTN respectively (P<0.001 compared to the diagonal reference line).

Using the univariate general linear model procedure on SPSS, and entering age as a covariate and gender and NYHA class as factors, analysis of the log normalised N-terminal proBNP levels in the heart failure patients yielded an r² of 0.446 for the model (P<0.001) with age, gender and NYHA class as significant predictive variables (P<0.034, 0.002 and 0.001 respectively). None of these factors was identified as predictive variables of UTN (r²=0.058). Thus, UTN levels in heart failure patients are elevated irrespective of age, gender or NYHA class.

From the comparison of the graphs in FIG. 2, one may see that the measurement of plasma UTN in combination with that of plasma NT proBNP is able to yield greater information concerning the diagnosis of heart failure than measurement of NT proBNP alone. Namely, levels of NT ProBNP only start to become elevated upon progression to NYHA Class 3, whereas plasma UTN levels are shown to be elevated in patients with an NYHA class of greater than 1. Thus, measurement of these two markers can lead to early stage identification of heart disease.

Equivalents

The present invention provides in part methods of diagnosing heart failure in a mammalian subject by measuring the levels of urotensin in a sample of bodily fluid derived from a subject. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

1. A method for detecting an increased risk of heart failure in a mammalian subject by detecting the level of urotensin in a bodily fluid sample whereby an elevated level of urotensin relative to the normal level is indicative of an increased risk of heart failure.

2. The method of claim 1, wherein the heart failure is the result of ischaemic cardiomyopathy, dilated cardiomyopathy, hypertensive cardiomyopathy or valvular disease.

3. The method of claim 1, wherein the level of urotensin is determined by using an immunoassay.

4. The method of claim 1, wherein the immunoassay is a lateral flow immunoassay.

5. The methods of claim 1, wherein the immunoassay is a flow-through immunoassay.

6. The method of claim 1, wherein the normal level is a level typically found in the said bodily fluid of a subject which is indicative of the absence of heart failure.

7. The method of claim 1, which comprises detecting a second marker whereby an elevated level of a second marker is indicative of an increased risk of heart failure.

8. The method of claim 7, wherein the second marker is NT-proBNP

9. The method of claim 7, wherein the second marker is BNP.

10. The method of claim 7, wherein the level of second marker is determined by use of an immunoassay.

11. The method of claim 1, wherein the immunoassay is a lateral flow immunoassay.

12. The methods of claim 1, wherein the immunoassay is a flow-through immunoassay.

13. The method of claim 7, wherein the bodily fluid is plasma.

14. The method of claim 7, wherein the bodily fluid is interstitial fluid.

15. The method of claim 7, wherein the subject is human.

16. The method of claim 7, wherein the level of urotensin is monitored periodically.

17. The method of claim 7, wherein the level of a second marker is monitored periodically.

18. A kit for the detecting an increased risk for heart failure in a subject, comprising an antibody for detecting a level of urotensin in a bodily fluid sample obtained from a subject.

19. The kit of claim 18, further comprising an antibody for measuring the level of a second marker.

20. The kit of claim 18, wherein the second marker is NT-proBNP

21. The kit of claim 18, wherein the second marker is BNP.
